(19) United States
(12) Patent Application Publication
(10) Pub. No.: US 2026/0335041 A1
(43) Pub. Date: Oct. 29, 2026

(54) APPARATUS, PLANT AND METHOD FOR THE INDUSTRIAL WASHING OF SURGICAL INSTRUMENTS

(71) Applicant: STEELCO S.P.A., RIESE PIO X (TV) (IT)

(72) Inventors: Alessandro CAPRARA, ISOLA DELLA SCALA (VR) (IT); Paolo RIGHETTINI, DALMINE (BG) (IT)

(73) Assignee: STEELCO S.P.A., RIESE PIO X (TV) (IT)

(21) Appl. No.: 18/998,863

(22) PCT Filed: Jul. 25, 2023
(86) PCT No.: PCT/IT2023/050174

§ 371 Date: Jan. 27, 2025

(30) Foreign Application Priority Data
Jul. 27, 2022 (IT) 102022000014011

(51) Int. Cl. A61B 90/70; A61L 2/04; A61L 103/15; B08B 3/04; B25J 11/00

(52) U.S. Cl. CPC A61B 90/70; A61L 2/04; B08B 3/04; B25J 11/0085; A61L 2103/15

(57) ABSTRACT

Washing apparatus (10) comprising at least one washing unit (15) provided with at least one washing station (55) for carrying out a washing operation on the surgical instruments (11) and manipulation means (19) for moving the surgical instruments (11) with respect to the at least one washing station (55), movement means (16) defining a feed line (L1) for moving said surgical instruments (11) to be washed toward the washing unit (15), and a pick-up line (L2) which is autonomous with respect to the feed line (L1) and configured to pick up the washed surgical instruments (11) from the at least one washing unit (15).

### FIELD OF THE INVENTION

The present invention concerns an apparatus, a plant and a method for the industrial washing of surgical instruments, for example coming from one or more operating theaters.

### BACKGROUND OF THE INVENTION

The instruments used during surgical operations are normally supplied to the medical team in kits, or sets, each comprising a determinate number and type of surgical instruments. For example, each kit contains instruments dedicated to a specific surgical procedure.

Normally, to avoid contamination during the washing and sterilization steps, a specific kit must never be dismantled, so that at the end of the wash a kit always contains the same identical instruments contained at the end of the surgical operation.

Usually, surgical instruments are washed in thermal disinfection washing machines, or instrument washers, and they are sterilized in steam sterilizers, for example autoclaves.

In addition, before the actual washing of the surgical instruments in the instrument washers, they are subjected to a preliminary wash with the aim of removing any solid residues of blood, or other material deriving from the surgical operation, deposited on the surgical instruments to be washed.

The preliminary wash provides to subject the instruments to a cold wash, to an ultrasonic wash and possibly also to a manual wash using manual apparatuses such as for example brushes to remove coagulated blood or other material. In fact, usually, not all the instruments coming from an operating theater have been used and need a thorough preliminary wash.

In the preliminary wash, an operator visually selects the surgical instruments and chooses whether to wash each of them in a cold washing machine, or to wash them manually.

This solution, although widely used, is not very efficient and productive, especially when it is required to wash a large number of instruments, for example in hospitals or surgeries which perform many surgical operations per day.

Document US2018/318458A1 describes a structure, an unmanned vehicle and a method for processing dirty surgical instruments.

Document WO2021/229614A1 concerns a washing apparatus and method.

Document US2021/145537A1 describes a system for washing and inspecting medical equipment.

Document ITU020100227A1 describes a perfected washing plant.

Document US2011/262250A1 describes an automated robotic system for handling surgical instruments.

Document WO2019/054910A1 describes a system for cleaning and disinfecting endoscopes.

Document EP3009201A1 describes a washing apparatus, in particular a thermal disinfection apparatus.

There is therefore a need to perfect an apparatus, a plant and a method for the industrial washing of surgical instruments, which can overcome at least one of the disadvantages of the state of the art.

To do this it is necessary to solve the technical problem of being able to automate the passage of the instruments from one washing machine to another, at the same time guaranteeing a high level of cleanliness.

In particular, one purpose of the present invention is to provide an apparatus, a plant and a method for the industrial washing of surgical instruments which can reduce the time required for washing them.

Another purpose of the present invention is to provide an apparatus, a plant and a method for the industrial washing of surgical instruments which does not require the presence of specialized personnel.

Another purpose of the present invention is to provide an apparatus, a plant and a method for the industrial washing of surgical instruments which is reliable and easy to use.

Another purpose of the present invention is to provide an apparatus, a plant and a method for the industrial washing of surgical instruments which allows to wash the surgical instruments without dismantling the kits to which they belong, and to trace the instruments which make up the kits.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes and to resolve the technical problem disclosed above in a new and original way, also achieving considerable advantages compared to the state of the prior art, an apparatus for washing surgical instruments, according to the present invention, comprises at least one washing unit for washing the surgical instruments and movement means defining a feed line which is configured to move the surgical instruments to be washed toward the at least one washing unit, and a pick-up line which is autonomous with respect to the feed line and is configured to pick up the surgical instruments from the at least one washing unit. The washing unit comprises at least one washing station and manipulation means configured to manipulate the surgical instruments from the movement means to the washing station, and vice versa.

According to one aspect of the invention, the manipulation means comprise at least one robotized anthropomorphic arm for moving the surgical instruments between the movement means and the washing station and for positioning the washed surgical instruments in the pick-up line. The washing apparatus further comprises an inlet station provided with at least one additional robotized anthropomorphic arm for positioning the instruments to be washed on the feed line.

In accordance with another aspect of the present invention, the movement means comprise a first movement member defining the feed line and a second movement member defining the pick-up line.

In accordance with another aspect of the present invention, the first movement member is disposed to one side of the manipulation means and the second movement member is disposed to a second side of the manipulation means, which is opposite to the first side.

According to another aspect of the invention, the washing apparatus also comprises first containers where the instruments to be washed are inserted before being positioned on the feed line and second containers positioned on the pick-up line and able to contain the washed instruments.

According to another aspect of the invention, the washing apparatus comprises an outlet station provided with a zone for grouping at least the second containers and also provided with at least one robotized anthropomorphic arm configured to pick up at least the second containers and position them on a mobile support member.

In accordance with another aspect of the present invention, the apparatus comprises a plurality of washing units aligned along an alignment axis.

In accordance with another aspect of the present invention, the second movement member is configured to move the second containers along the pick-up line.

In accordance with another aspect of the present invention, the at least one washing unit comprises at least a recognition station configured to recognize the surgical instruments, a detection station configured to detect organic traces on the surgical instruments. The at least one robotized anthropomorphic arm is configured to move the surgical instruments between the movement means and the stations.

In accordance with another aspect of the present invention, the at least one washing unit comprises a manipulation station comprising additional manipulation means which are configured to cooperate with the manipulation means. The additional manipulation means comprise a rest plane on which hooking elements are attached, on which it is possible to hook the instruments by means of the manipulation means, therefore by means of the robotized anthropomorphic arm.

According to another aspect of the invention, at least the movement means and the washing unit are controlled by a central control unit which manages both the movement of the instruments in the apparatus and also the operation of the washing unit.

According to another aspect of the invention, with each first container there is associated a respective recognition element encoding the information on the instruments contained therein. The apparatus comprises a feed zone provided with recognition means configured to recognize the recognition element the instant in which the first container is positioned on the feed line.

According to another aspect of the invention, the washing apparatus comprises a recognition station configured to recognize the instruments of a group of instruments and provided with at least a first viewing device connected to a unit for processing the images taken by the viewing device.

The present invention also concerns a plant for washing surgical instruments comprising an apparatus according to the present invention, at least one machine for the thermal disinfection of the surgical instruments and additional movement means configured to automatically move the surgical instruments coming out of the apparatus toward the at least one thermal disinfection machine.

In accordance with another aspect of the present invention, a method for washing surgical instruments by means of a washing apparatus according to the present invention comprises the following steps:
a feed step in which the surgical instruments to be washed are fed along the feed line toward the at least one washing unit by means of the additional robotized anthropomorphic arm;
a washing step in which the manipulation means, by means of the robotized anthropomorphic arm, take one of the tools into the washing station to perform a washing operation;
a pick-up step in which the surgical instrument, washed in the washing station, is picked up from the washing unit and disposed on the pick-up line, by means of the robotized anthropomorphic arm.

In accordance with another aspect of the present invention, the feed step also provides to feed first containers, in which the instruments to be washed are inserted before being positioned on the feed line, and second containers, which are positioned on the pick-up line and are able to contain the washed instruments.

### DESCRIPTION OF THE DRAWINGS

These and other aspects, characteristics and advantages of the present invention will become apparent from the following description of some embodiments, given as a non-restrictive example with reference to the attached drawings wherein:

FIGS. 1 and 2 are schematic plan views of two washing apparatuses in accordance with two embodiments of the present invention;

FIGS. 3 and 4 are three-dimensional views of respective portions of the apparatuses of FIGS. 1 and 2, respectively;

FIGS. 5, 6 and 7 are split three-dimensional views of three parts of an apparatus according to the present invention;

FIG. 8 is an electrical diagram of an apparatus according to the present invention;

FIG. 9 is a schematic top view of a washing plant according to the present invention.

We must clarify that in the present description the phraseology and terminology used, as well as the figures in the attached drawings also as described, have the sole function of better illustrating and explaining the present invention, their function being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

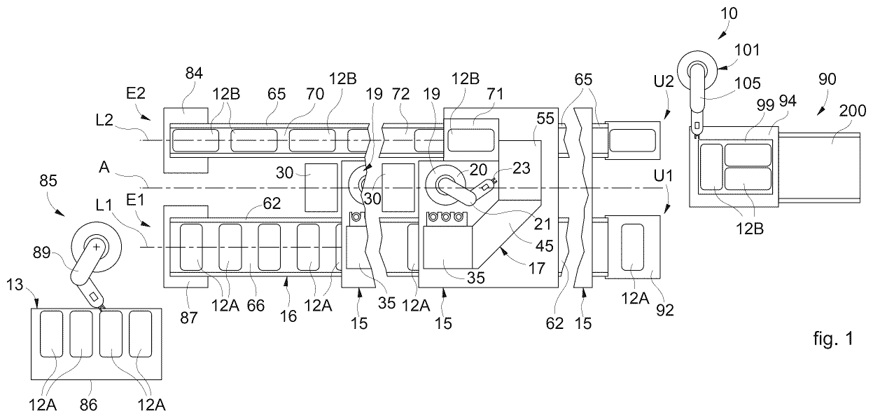

With reference to FIG. 1, an apparatus 10 according to the present invention is configured to automatically perform a wash, for example a preliminary wash, of surgical instruments 11, hereafter instruments 11.

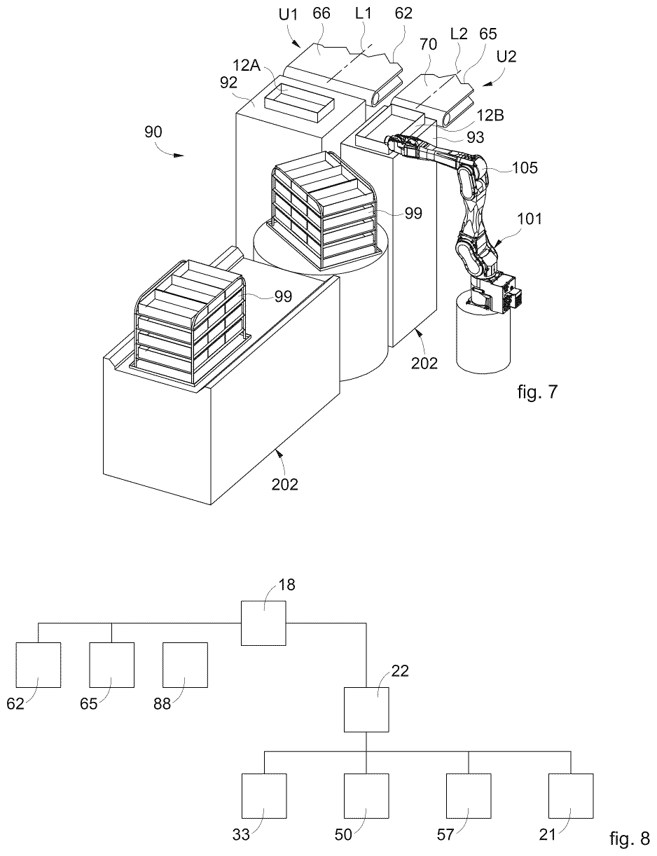

Preferably, the apparatus 10 of the present invention is inserted in a washing plant 200 (FIG. 7), upstream of a thermal disinfection machine 201 configured to treat the instruments 11 washed by means of the apparatus 10 of the present invention.

The instruments 11 to be washed come from one or more operating theatres and are grouped into kits 13 (FIG. 1), or sets, of instruments 11. In particular, here and hereafter in the description, with the term “kit” 13 we mean a certain group of instruments 11.

Optionally, the apparatus 10 also comprises a plurality of containers 12 and the instruments 11 of each kit 13 are divided into one or more containers 12, which are open at the top and provided with holes to allow the instruments 11 contained therein to be washed.

For example, a container 12 can be made of metal and its walls can be conformed as a mesh, or a net, in order to define a plurality of passage apertures for water or other washing fluids.

With each container 12 there is associated a respective recognition element 14 encoding the information on the instruments 11 contained therein. The information can also concern the kit 13 to which the instruments 11 belong.

Purely by way of example, the recognition element 14 can be of the barcode, QR code, RFID, Bluetooth or other type, configured to transmit data through electromagnetic waves, or similar and comparable types.

With reference to FIG. 1, the apparatus 10 comprises movement means 16 defining both a feed line L1 having an inlet E1 and an outlet U1, and also a pick-up line L2 having an inlet E2 and an outlet U2, and a plurality of washing units 15 associated with the movement means 16.

The movement means 16 are configured both to feed groups of instruments 11 to be washed toward the washing units 15, and also to pick up and move away the groups of washed instruments 11 from the washing units 15.

In particular, the feed line L1 is configured to feed the instruments to be washed from the inlet E1 toward the washing units 15, and the pick-up line L2 is configured to move the washed instruments 11 from the washing units 15 toward the outlet end U2.

We must clarify that the movement means 16 can feed a plurality of groups of instruments 11 substantially simultaneously toward the washing units 15 and along the feed line L1. In this case, each group of instruments 11 is moved toward a respective washing unit 15.

The movement means 16 and each washing unit 15 are controlled by a central control unit 18 (FIG. 8) which manages both the movement of the instruments 11 in the apparatus 10 and also the operation of each washing unit 15.

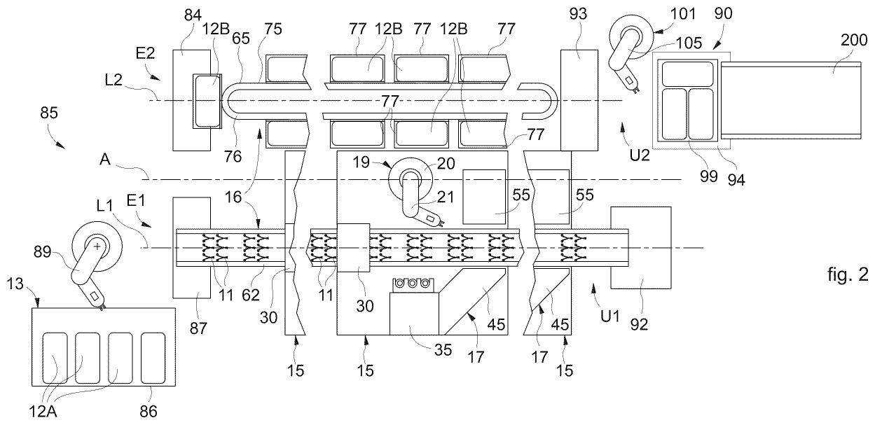

In the example given here, the washing units 15 are aligned along an alignment axis A (FIGS. 1 and 2) and each of them comprises both a plurality of operating stations 17 and also manipulation means 19 configured to move the instruments 11 between the operating stations 17, in particular the washing stations 55, and the movement means 16.

In particular, the manipulation means 19 are configured to pick up the instruments 11 to be washed from the feed line L1 and to position the washed instruments 11 in the pick-up line L2.

In this specific case, the manipulation means 19 (FIGS. from 1 to 4) comprise at least one support structure 20 on which a robotized anthropomorphic arm 21 is attached. The robotized anthropomorphic arm 21 is commanded by a local control unit 22 connected to the central control unit 18.

At one end of the robotized anthropomorphic arm 21 there is attached a gripping tool 23 suitable to grip the instruments 11 and which, by way of example, can comprise a gripper.

The robotized anthropomorphic arm 21 is sized so as to be able to reach both the feed line L1 as well as the pick-up line L2 and each operating station 17 of the respective washing unit 15, in particular the washing station 55.

In the example given here, each washing unit 15 (FIGS. from 1 to 4) comprises a recognition station 30, a manipulation station 40, a detection station 50 and the washing station 55.

The recognition station 30 is configured to recognize the instruments 11 of a group of instruments 11. The recognition station 30 is configured to communicate with the local control unit 22 so as to slave the control of the manipulation means 19.

In particular, the recognition station 30 comprises at least a first viewing device 32, such as a video camera or suchlike, connected to a unit 33 for processing the images taken by the viewing device 32. The viewing device 32 can be positioned in a support structure 31 located in the vicinity of the feed line L1 and of the robotized anthropomorphic arm 21.

During use, the first viewing device 32 captures one or more images of the group of instruments 11 to be washed which is fed to the washing unit 15, and the processing unit 33 is configured to determine the relative position between the manipulation means 19 and the instruments 11 to be washed.

Furthermore, the processing unit 33 is also configured to transmit an electrical signal to the local control unit 22 encoding the relative position between the manipulation means 19 and the instruments 11 to be washed.

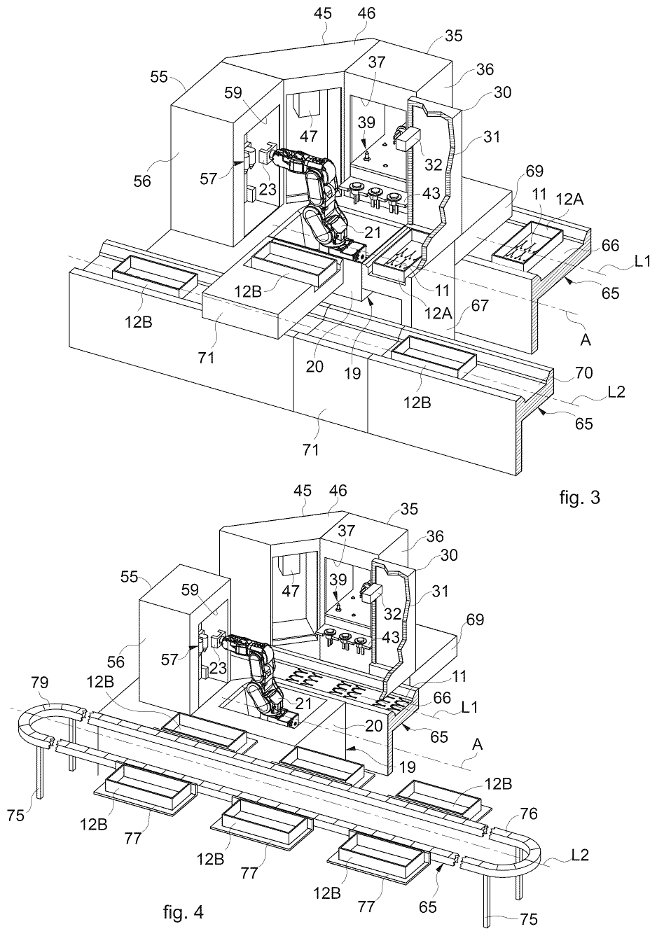

The manipulation station 35 (FIGS. 3, 4 and 6) comprises an external structure 36 having a front aperture 37 inside which second manipulation means 39 are disposed.

The second manipulation means 39 comprise a rest plane 40 on which hooking elements 41 are attached, which are configured to hook onto the instruments 11. The instruments 11 are hooked to the hooking elements 41 by means of the manipulation means 19, therefore by means of the robotized anthropomorphic arm 21.

Purely by way of example, the hooking elements 41 can comprise two vertical pegs 42, each of which is configured to be inserted into a respective ring of surgical scissors, so as to be able to open them in order to expose the entire surface of the blades.

In addition, a temporary retaining member 43 for a plurality of gripping tools 23 which are configured to be connected to the end of the robotized anthropomorphic arm 51 can be disposed inside the manipulation station 35.

The detection station 45 (FIGS. 3 and 4) comprises an external structure 46 containing a second viewing device 47 and having a front aperture 49 that allows the robotized anthropomorphic arm 51 to insert an instrument 11 in the proximity of the second viewing device 47.

The second viewing device 47 is connected to a processing unit 50 for processing the images taken by the second viewing device 47 and detecting any organic traces on the instruments 11.

Moreover, the processing unit 50 is also configured to determine the relative position of the organic traces detected on the instruments 11 with respect to a reference of the manipulation means 19 and to transmit an electrical signal encoding the relative position to the local control unit 22 in order to slave the washing of the instrument 11.

Purely by way of example, the viewing device 47 comprises a light emitter and a sensor (not shown) for receiving the reflected light, and possibly also an optic for conveying the reflected light toward the receiving sensor.

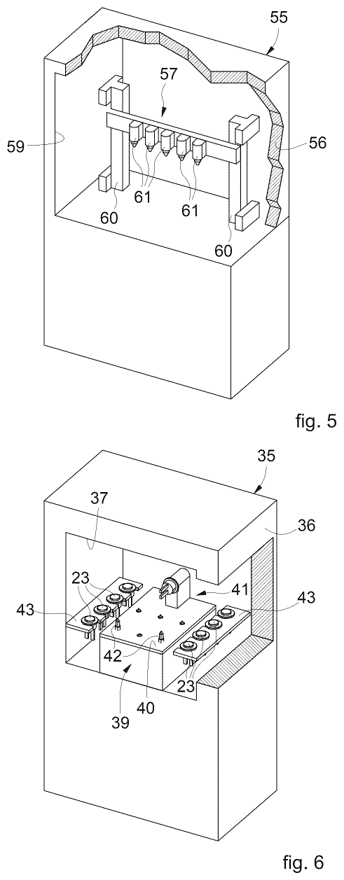

The washing station 55 (FIGS. 3, 4 and 5) comprises an external structure 56 containing a washing device 57 and having a front aperture 59 for inserting the instruments 11 to be washed.

The washing device 57 is configured to deliver a washing liquid on the instruments 11 to be washed and, in the example given here, it comprises a frame 60 to which one or more nozzles 61 are attached, in the present case five, each of which is connected to a respective hydraulic circuit (not shown) that feeds a specific washing fluid. Purely by way of example, the washing fluids can comprise: water, water and bicarbonate, steam, hydrogen peroxide and others.

Each nozzle 61 can be driven by the local control unit 22 and emits a jet of washing fluid for washing an instrument 11 disposed below it by the manipulation means, for example as a function of the position of the organic traces on the instrument 11.

The person of skill in the art understands, however, that the washing device 57 can also be very different from what has been described so far, and it can also be selected from washing devices known per se, depending on the productivity required of the apparatus 10 and also on the desired washing procedure.

The movement means 16 comprise a first movement member 62 defining the feed line L1 along which the instruments 11 to be washed are fed toward the washing units 15.

The first movement member 62 is commanded by the central control unit 18 and is configured to make the instruments 11 to be washed advance along the feed line L1, toward one of the washing units 15.

Furthermore, the movement means 16 also comprise a second movement member 65 defining the pick-up line L2 along which the washed instruments 11 are moved toward the outlet end U2 in order to be picked up by the apparatus 10.

Preferably, the first movement member 62 is disposed to a first side of the manipulation means 19 of the washing units 15, while the second movement member 65 is disposed to a second side of the manipulation means 19 of the washing units 15, opposite to the first side.

In a first embodiment, schematically shown in FIGS. 1 and 3, the instruments 11 to be washed are disposed in groups inside a plurality of first containers 12A which are positioned on the first movement member 62.

In this case, the first movement member 62 comprises a conveyor belt 66 parallel to the alignment axis A of the washing units 15 and disposed in the proximity thereof, for example below them. Moreover, the first movement member 62 also comprises a plurality of first supports 67, each of which is associated with a respective washing unit 15 and is configured to support one of the first containers 12A in proximity to the manipulation means 19 of the washing unit 15 with which it is associated.

The first movement member 62 also comprises a plurality of transfer devices 69, each of which is associated with one of the first supports 67 and with the conveyor belt 66, and it is configured to transfer one of the first containers 12A from the conveyor belt 66 to the first support 69 with which it is associated, and vice versa.

Furthermore, in this first embodiment, the second movement member 65 comprises a second conveyor belt 70 which develops parallel to the alignment axis A of the washing units 15 and in the proximity thereof.

The second movement member 65 also comprises a plurality of second supports 71, each of which is associated with a respective washing unit 15 and is configured to support a second container 12B.

The second movement member 62 also comprises a plurality of transfer devices 72, each of which is associated with one of the second supports 71 and also with the second conveyor belt 70, and it is configured to transfer one of the second containers 12B from the second conveyor belt 70 to one of the second supports 71, and vice versa.

In a second embodiment, schematically shown in FIGS. 2 and 4, the first conveyor belt 66 develops parallel to the alignment axis A of the washing units 15 and is disposed in proximity to the manipulation means 19, at a height such that the latter can easily reach the instruments 11 to be washed disposed thereon.

For example, the conveyor belt 66 can be disposed made through between the detection station 45 and the washing station 55, and directly below the recognition station 30 of each washing unit 15.

In this embodiment, the instruments 11 to be washed can be disposed directly on the first movement member 62 without them necessarily being inside the first containers 12A, which in this embodiment are not used.

In fact, since the first movement member 62 is disposed in proximity to the manipulation means 19, the latter can reach the tools 11 to be washed disposed thereon.

In this second embodiment, the second movement member 65 comprises a structure 75 on which there is attached a guide 76 which defines the pick-up line L2 and on which one or more transport elements 77, mobile independently of each other, are mounted.

Purely by way of example, the guide 76 can comprise first magnetic means 79, which can be selectively activated even independently of each other, and each transport element comprises second magnetic means 80 which, in response to the activation of the first magnetic means 79, cause the translation of the respective transport element 77 along the guide.

Each transport element 77 is configured to support a second container 12B and to move it with respect to the manipulation means 19 of one of the washing units 15.

The apparatus 10 also comprises an inlet station 85 disposed in particular in proximity to the inlet ends E1, E2 of the lines L1, L2 and configured to allow the positioning of the instruments 11 to be washed on the feed line L1. The apparatus 10 comprises containers 12B for the washed instruments 11 positioned on the pick-up line L2. The instruments 11 to be washed are positioned on the first movement member 62 and the second containers 12B on the second movement member 65.

The inlet station 85 comprises both a collection zone 86 in which the instruments 11 to be washed are disposed, and also a first feed zone 87 disposed in correspondence with the inlet end E1 of the first movement member 62.

In the first embodiment, the instruments 11 to be washed are disposed inside first containers 12A which are disposed on the first movement member 62.

Instead, in the second embodiment, the instruments 11 to be washed are supplied to the collection zone 86 inside first containers 12A, which are emptied onto the first movement member 62 which, in this case, will only transport the instruments 11 to be washed.

Optionally, in the first embodiment, the first feed zone 87 also comprises recognition means 88 configured to recognize a recognition element 14 associated with each first container 12A the instant in which the latter is positioned on the feed line L1, in particular on the first movement member 62. The recognition means 88 can be devices known per se and chosen according to the type of recognition element 14 with which the container 12a is equipped, therefore they can for example provide barcode, QR code, RFID or Bluetooth readers, or other types of readers.

The recognition means 88 are also configured to transmit information to the central control unit 18 relating to the instant in which a first container 12A is positioned on the first movement member 62, in order to allow to determine the next position of the first container 12A considered.

In this way, the central control unit 18 can process the information on the instant in which a first container 12A is positioned on the first movement member 62 and the extent of the movement commanded to the latter, so as to be able to determine the position of each container 12A present on the feed line L1.

The transfer of the instruments to be washed from the collection zone 86 to the first feed zone 87 can be performed manually by a user U.

Preferably, the inlet station 85 comprises at least one robotized anthropomorphic arm 89 (FIG. 1) configured to pick up the surgical instruments 11 from the inlet station 85 and to transfer them onto the first feed line L1. In particular, the robotized anthropomorphic arm 89 is configured to pick up at least one of the first containers 12A from the collection zone 86 and to position it, or to position the instruments 11 contained therein, on the first movement member 62.

Optionally, in the first embodiment, the inlet station 85 also comprises a second feed zone 84 disposed in correspondence with the inlet end E2 of the second movement member 65 and configured to allow the positioning of second empty containers 12B on the second movement member 65.

The apparatus 10 also comprises an outlet station 90 (FIGS. 1, 2 and 7) disposed in correspondence with the outlet ends U1, U2 and configured to allow to pick up the washed instruments 11 from the pick-up line L2.

Moreover, the outlet station 90 is also configured to allow to pick up any unprocessed instruments 11, or first containers 12A, from the feed line L1.

For example, in the first embodiment, the first movement member can transport the first containers 12A up to the outlet station 90, which are empty or contain instruments 11 not processed by the washing units 15.

Alternatively, in the second embodiment, the second movement member 65 can transport instruments 11 not processed by the washing units 15 to the outlet station 90.

The instruments 11 transported by the first movement member 62 to the outlet station 90 and not processed by the washing units 15 are instruments 11 which, for any reason, for example size or type, the washing units 15 have failed to process.

The outlet station 90 comprises a first extraction zone 92, disposed in correspondence with the outlet end U1 of the feed line L1 in which the instruments 11, or the first empty or not empty containers 12A, pushed out by the first movement member 62 are left, and a second extraction zone 93 disposed in correspondence with the outlet end U2 of the pick-up line L2 in which the second containers 12B containing the washed instruments 11 are disposed.

Optionally, the outlet station 90 also comprises a grouping zone 94 of the second containers 12B. The second containers 12B are present in the second extraction zone 93 and are disposed on a mobile support member 99, for example a trolley having a plurality of rest planes, which is configured to be inserted in a machine 201 for the thermal disinfection of the instruments 11, which is disposed downstream of the apparatus 10.

The transfer of the containers 12 from the extraction zone 93 to the grouping zone 94 occurs manually and is performed by a user who positions each container 12 in a mobile support member 99.

Alternatively, the outlet station 90 comprises grouping means 101 (FIGS. 1, 2 and 7) configured to position the containers 12B containing the washed instruments on the mobile support member 99. Each container 12B present in the second extraction zone 93 is positioned on the mobile support member 99 disposed in the grouping zone 94.

In the example given here, the grouping means 101 comprise at least one robotized anthropomorphic arm 105 configured to pick up at least the containers 12B and position them on the mobile support member 99. These containers 12B are positioned in the second extraction zone 99.

A person of skill in the art readily understands that the characteristics of the embodiments described here can be combined with each other in order to create further embodiments that fall within the scope of the present invention.

For example, in a third embodiment the apparatus could comprise a first movement member as in the first embodiment described here, and a second movement member as in the second embodiment described here.

Alternatively, in a fourth embodiment the apparatus could comprise a first movement member as in the second embodiment described here, and a second movement member as in the first embodiment described here.

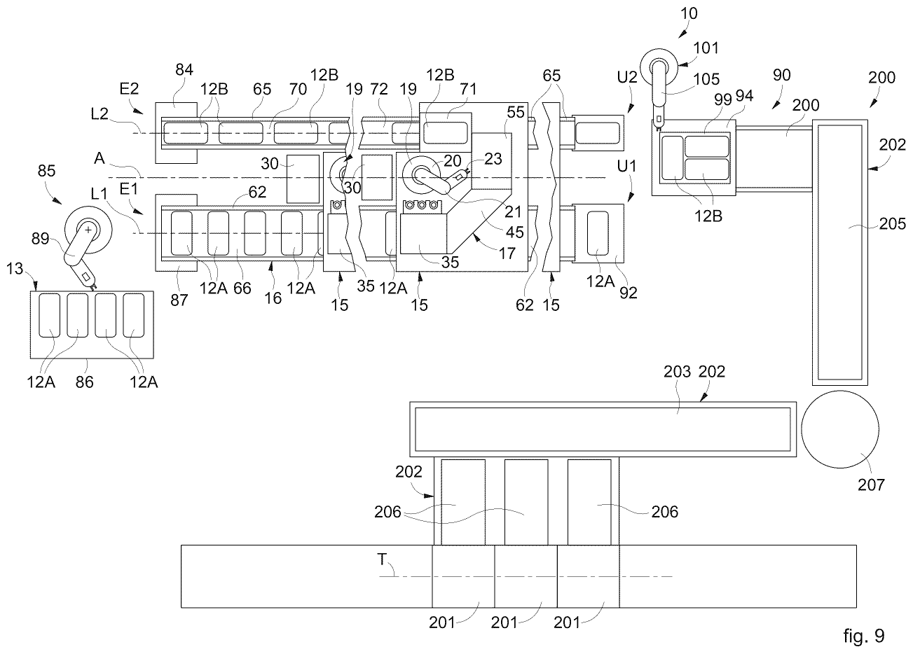

The present invention also concerns a plant 200 (FIG. 9) for washing surgical instruments 11, which comprises an apparatus 10 as described above and at least one thermal disinfection machine 201 disposed downstream of the apparatus 10.

Preferably, the plant 200 comprises a plurality of thermal disinfection machines 201 aligned along an alignment axis T, and transport means 202 defining a first transport segment 203 parallel to the alignment axis T and a second transport segment 205 running from the outlet station 90 of the apparatus 10 of the present invention to the beginning of the first transport segment 203.

Optionally, in front of each thermal disinfection machine 201 there is disposed another insertion segment 206 of the transport means 202, which is configured to insert in the respective thermal disinfection machine 201 a mobile support member 99 transiting on the first transport segment 103.

In addition, between the first 203 and the second transport segment 205 there is disposed a platform 207 configured to allow the mobile supports 99 that transit on the transport means 202 to change direction.

The operation of the apparatus 10 described heretofore, which corresponds to the method according to the present invention, comprises the following steps.

The method provides an introduction step in which the instruments 11 to be washed are positioned on the first movement member 62 and second empty containers 12B are positioned on the second movement member 65.

In the first embodiment, the instruments 11 to be washed are contained in first containers 12A which are disposed on the first movement member 62.

Alternatively, in the second embodiment, the instruments 11 to be washed are disposed directly on the first movement member 62.

This introduction step can be performed manually by an operator, who positions the instruments 11 on the first movement member 62.

Alternatively, in the embodiments which provide it, this introduction step can be performed automatically by a robotized anthropomorphic arm 89.

Optionally, in the first embodiment, when a first container 12A is positioned on the first movement member 62, a recognition step also occurs, in which the recognition means 88 recognize the recognition element 14 associated with that determinate first container 12A and transmit an electrical signal to the control unit 18 comprising the information contained in the recognition element 14 and temporal information relating to the instant in which the container 12 was positioned on the first movement member 62.

Subsequently, the method provides a first feed step in which the instruments 11 to be washed are made to advance along the feed line L1 and the second empty containers 12B are made to advance along the pick-up line L2.

The advance of the instruments 11 to be washed and of the second containers 12B along the feed line L1 and the pick-up line L2, respectively, is commanded by the central control unit 18 which in turn commands the movement members 62,65.

For example, the advance can be stepwise or continuous.

Alternatively, in the second embodiment, the second containers 12B are moved independently along the guide 76 of the second movement member 65.

The method also provides a stop step in which the first movement member 62 is stopped, so that a group of instruments 11 to be washed is in the proximity of one of the washing units 15.

In addition, the stop step also provides to stop the second movement member 65 so that a second empty container 12B is in the proximity of one of the washing units 15.

In the first embodiment, there is also provided a pick-up step in which a first container 12A containing the instruments 11 to be washed is automatically picked up by the first movement member 62 and positioned on the first support 67 associated with the washing unit 15.

In this case, the pick-up step also provides to automatically pick up a second empty container 12B from the second movement member 65 and position it on the second support 71 associated with the washing unit 15.

Then, the method provides a recognition step in which the instruments 11 to be washed are located below in the recognition station 30.

The recognition step provides to recognize the instruments 11 and transmit an electrical signal to the local control unit 22 encoding the relative position between the manipulation means 19 and the instruments 11 to be washed.

Subsequently, the method provides a detection step in which one of the instruments 11 to be washed is taken into the detection station 45 by the manipulation means 19 in order to detect any organic traces present on the instrument 11 and determine the relative position of the organic traces with respect to a reference point of the manipulation means 19.

Then, after the detection step, the method provides a washing step in which the manipulation means 19 take the instrument 11 into the washing station 55 and the washing device 57 is activated. Preferably, in this washing step the instrument 11 to be washed is positioned with respect to the washing device 57 in such a way as to expose to the latter the organic traces present on the instrument 11.

When the washing step is finished, the method provides a release step in which the manipulation means 19 release the washed instrument 11 into the second empty container 12B disposed on the second movement member 65.

Then the detection and washing steps are repeated for all the instruments 11 to be washed in the washing unit 15 under consideration.

Optionally, before the detection step, the method provides a manipulation step in which the manipulation means 19 take the instrument 11 inside the manipulation station 35 in order to modify its attitude, so as to facilitate the subsequent steps of detecting traces of dirt on the instrument 11 and washing.

Subsequently, the method provides a pick-up step in which the second container 12B is made to advance along the pick-up line L2 and is taken toward the second extraction zone 93 of the outlet station 90 from which it is extracted, manually or automatically, and positioned on the mobile support member 99.

Preferably, the mobile support member 99 will subsequently be used to take a plurality of containers 12 toward a thermal disinfection machine 201 disposed downstream of the apparatus 10, for example as in the plant 200 (FIG. 9) according to the present invention.

It is clear that modifications and/or additions of parts may be made to the apparatus 10, to the plant 200 and to the method for the industrial washing of surgical instruments 11 as described heretofore, without departing from the field and scope of the present invention, as defined by the claims.

It is also clear that, although the present invention has been described with reference to some specific examples, a person of skill in the art will be able to achieve other equivalent forms of apparatus 10, plant 200 and method for the industrial washing of surgical instruments 11, having the characteristics as set forth in the claims and hence all coming within the field of protection defined thereby. In the following claims, the sole purpose of the references in brackets is to facilitate their reading and they must not be considered as restrictive factors with regard to the field of protection defined by the very claims.

1: Washing apparatus (10) for washing surgical instruments (11), comprising at least one washing unit (15) for washing said surgical instruments (11) and movement means (16) defining a feed line (L1) which is configured to move said surgical instruments (11) to be washed toward said at least one washing unit (15), and a pick-up line (L2) which is autonomous with respect to the feed line (L1) and is configured to pick up said surgical instruments (11) from said at least one washing unit (15), wherein said washing unit (15) comprises at least one washing station (55) and manipulation means (19) configured to manipulate said surgical instruments (11) from said movement means (16) to said washing station (55), and vice versa, characterized in that said manipulation means (19) comprise at least one robotized anthropomorphic arm (21) for moving said surgical instruments (11) between said movement means (16) and said washing station (55) and for positioning said washed surgical instruments (11) in said pick-up line (L2), said washing apparatus (10) further comprising an inlet station (85) provided with at least one additional robotized anthropomorphic arm (89) for positioning the instruments (11) to be washed on said feed line (L1).

2: Washing apparatus (10) as in claim 1, characterized in that said movement means (16) comprise a first movement member (62) defining said feed line (L1) and a second movement member (65) defining said pick-up line (L2).

3: Washing apparatus (10) as in claim 2, characterized in that said first movement member (62) is disposed to one side of said manipulation means (19) and said second movement member (65) is disposed to a second side of said manipulation means (19), which is opposite to said first side.

4: Washing apparatus (10) as in claim 1, characterized in that it comprises first containers (12A) where the instruments (11) to be washed are inserted before being positioned on said feed line (L1) and second containers (12B) positioned on said pick-up line (L2) and able to contain the washed instruments (11).

5: Washing apparatus (10) as in claim 4, characterized in that it comprises an outlet station (90) provided with a zone (94) for grouping at least said second containers (12B) and also provided with at least one robotized anthropomorphic arm (105) configured to pick up at least said second containers (12B) and position them on a mobile support member (99).

6: Washing apparatus (10) as in claim 1, characterized in that it comprises a plurality of washing units (15) aligned along an alignment axis (A).

7: Washing apparatus (10) as in claim 2, characterized in that said second movement member (65) is configured to move said second containers (12B) along said pick-up line (L2).

8: Washing apparatus (10), as in claim 1, characterized in that said at least one washing unit (15) comprises at least a recognition station (30) configured to recognize said surgical instruments (11), a detection station configured to detect organic traces on said surgical instruments (11), said at least one robotized anthropomorphic arm (21) being configured to move said surgical instruments (11) between said movement means (16) and said stations (17, 30, 45).

9: Washing apparatus (10), as in claim 1, characterized in that said at least one washing unit (15) comprises a manipulation station (35) comprising additional manipulation means (39) which are configured to cooperate with said manipulation means (19), said additional manipulation means (39) comprising a rest plane (40) on which hooking elements (41) are attached, on which it is possible to hook said instruments (11) by means of said manipulation means (19), therefore by means of said robotized anthropomorphic arm (21).

10: Washing apparatus (10) as in claim 1, characterized in that at least said movement means (16) and said washing unit (15) are controlled by a central control unit (18) which manages both the movement of the instruments (11) in the apparatus (10) and also the operation of said washing unit (15).

11: Washing apparatus (10) as in claim 2, characterized in that with each first container (12A) there is associated a respective recognition element (14) encoding the information on the instruments (11) contained therein, said apparatus (10) comprising a feed zone (87) provided with recognition means (88) configured to recognize said recognition element (14) the instant in which said first container (12A) is positioned on said feed line (L1).

12: Washing apparatus (10) as in claim 1, characterized in that it comprises a recognition station (30) configured to recognize the instruments (11) of a group of instruments (11) and provided with at least a first viewing device (32) connected to a unit (33) for processing the images taken by the viewing device (32).

13: Plant (200) for washing surgical instruments (11) comprising an apparatus (10) as in claim 1, at least one machine (201) for the thermal disinfection of said surgical instruments (11) and additional movement means (202) configured to automatically move said surgical instruments (11) coming out of said apparatus (10) toward said at least one thermal disinfection machine (201).

14: Method for washing surgical instruments (11) by means of a washing apparatus (10) comprising at least one washing unit (15) provided with at least one washing station (55) which is configured to perform a washing operation on said surgical instruments (11) and manipulation means (19) configured to manipulate said surgical instruments (11) and move them with respect to said washing station (55), movement means (16) defining a feed line (L1) which is configured to move said surgical instruments (11) to be washed toward said at least one washing unit (15), and a pick-up line (L2) which is autonomous with respect to the feed line (L1) and is configured to pick up said washed surgical instruments (11), wherein said manipulation means (19) comprise at least one robotized anthropomorphic arm (21) for moving said surgical instruments (11) between said movement means (16) and said washing station (55) and for positioning said washed surgical instruments (11) in said pick-up line (L2), said washing apparatus (10) also comprising an inlet station (85) provided with at least one additional robotized anthropomorphic arm (89) for positioning the instruments (11) to be washed on said feed line (L1), said method providing the following steps:
a feed step in which said surgical instruments (11) to be washed are fed along said feed line (L1) toward said at least one washing unit (15) by means of said additional robotized anthropomorphic arm (89);
a washing step in which said manipulation means (19), by means of said robotized anthropomorphic arm (21), take one of said tools (11) into said washing station (55) to perform a washing operation;
a pick-up step in which said surgical instrument (11), washed in said washing station (55), is picked up from said washing unit (15) and disposed on said pick-up line (L2), by means of said robotized anthropomorphic arm (21).

15: Method as in claim 14, characterized in that said feed step also provides to feed first containers (12A), in which the instruments (11) to be washed are inserted before being positioned on said feed line (L1), and second containers (12B), which are positioned on said pick-up line (L2) and are able to contain the washed instruments (11).
